(19) United States
(12) Patent Application Publication
(10) Pub. No.: US 2004/0078230 A1
(43) Pub. Date: Apr. 22, 2004

(54) Managing a medical procedure

(76) Inventor: Johannis Andreas Karas, Pretoria (ZA)

Correspondence Address: ROTHWELL, FIGG, ERNST & MANBECK, P.C., 1425 K STREET, N.W., SUITE 800, WASHINGTON, DC, 20005, US

(21) Appl. No.: 10/450,385

(22) PCT Filed: Dec. 14, 2001
(86) PCT No.: PCT/IB01/02478

(30) Foreign Application Priority Data
Dec. 14, 2000 (ZA) 00/6605

(51) Int. Cl. G06F 17/60; A61B 10/00

(52) U.S. Cl. 705/2; 600/562

(57) ABSTRACT

The invention relates to a method of and a service facility for providing a pathological service for analysing an exfoliative cytological sample from fluid from reproductive organs of a female (pap smear). The method includes offering on the Internet by means of a server a pathological service as described, receiving and processing acceptance of the offer by a client, and making available to the client a do-it-yourself kit (10) for obtaining a cytological sample as described. The method further includes receiving the sample, analysing the sample pathologically, and communicating the results of the analysis. The invention extends to a do-it-yourself kit (10) for obtaining an exfoliative cytological sample from fluid from reproductive organs of a female (pap smear).

THIS INVENTION relates to managing a medical procedure. It relates more specifically to a method of and a service facility for providing a pathological service for analysing an exfoliative cytological sample from fluid from reproductive organs of a female (colloquially known as a “pap smear”), and to a do-it-yourself kit for use in carrying out the method.

In accordance with one aspect of the invention, there is provided a method of providing a pathological service for analysing an exfoliative cytological sample from fluid from reproductive organs of a female (pap smear), including

offering on the internut by means of a server a pathological service as described;

receiving and processing acceptance of the offer by a client;

making available to the client a do-it-yourself kit for obtaining a cytological sample as described;

receiving the sample;

analysing the sample pathologically; and

communicating the result of the analysis.

Offering of the service on the Internet may be via a website,

The invention extends to a method of providing a pathological service for analysing an exfoliative cytological sample from fluid from reproductive organs of a female (pap smear), including

making available to a client a do-it-yourself kit for obtaining a cytological sample as described;

receiving the sample;

analysing the sample pathologically; and

communicating the result of the analysis.

Receiving and processing acceptance of the offer by the client may be by means of an interactive procedure managed by software provided for this purpose in the server.

The method may include allocating to the client a personal identification number (PIN) to ensure confidentiality.

Communicating the result of the analysis may include making the result available via the server in response to receipt of the PIN.

Receiving acceptance of the offer may be personally by telephone, short message service (SMS), facsimile, Internet, email, post, or in person. Similarly, the result of the analysis may be personally communicated by telephone, SMS, facsimile, Internet, e-mail, post, or in person.

The method may include receiving and processing an instruction to provide the result to an interested third party, e.g. a medical practitioner.

The making available to the client of the do-it-yourself kit 5 may be by means of online commerce. Instead, the making available to the client of the do-it-yourself kit may be by means of direct retail or mail order.

Analysing the sample pathologically may include conducting a pap smear test, a Human Papilloma Virus test, and a microbiological and molecular screen of organisms present in the sample.

In accordance with another aspect of the invention, there is provided a service facility for providing a pathological service for analysing an exfoliative cytological sample from fluid from reproductive organs of a female (pap smear), the service facility including

a server accessible via the Internet by a client computer, the server having a database incorporating information about the service and having an interactive facility for use by the client computer to obtain a do-it-yourself kit for obtaining a sample as described;

a stock of do-it-yourself kits for obtaining samples as described;

a receiving unit for receiving a sample;

a pathological analysis unit for pathologically analysing the sample; and

a processing and communicating facility of the server for processing a result of the pathological analysis and communicating the result to the client computer via a datalink.

The server may make the database and the interactive facility available via an Internet website.

The interactive facility may include software for receiving particulars of a credit card or like electronic payment means.

The interactive facility may have software for allocating a personal identification number (PIN) to a client computer or for receiving a PIN selected by the client and for keeping information about the client confidential against appropriate use of the PIN.

The software may be usable to allow verification by means of the PIN for providing a do-it-yourself kit.

The database may include instructions for instructing a client to obtain a sample. The instructions may be in the form of an animation sequence, which may be downloadable to the client computer.

In accordance with yet another aspect of the invention, there is provided a do-it-yourself kit for obtaining an exfoliative cytological sample from fluid from reproductive organs of a female (pap smear), the do-it-yourself kit including

a holder;

user instructions in or on the holder for using the kit; and

a sealable container in which a tampon is receivable and which contains a preserving liquid for preserving cellular, bacterial and fungal matter on the tampon after the sample has been obtained, the container being receivable in the holder.

The do-it-yourself kit may include an information sheet for receiving personal information of a user of the kit, and may be used in a method of providing a pathological service as hereinbefore described.

The preserving liquid may include a pH buffer. The preserving liquid may also include antibiotics. Furthermore, the preserving liquid may include an anti-clumping agent.

The holder may include securing means for securing the container in position inside the holder.

The do-it-yourself kit may include a tampon.

If desired, the do-it-yourself kit may include at least one microscope slide and a fixative for fixing a sample smeared onto the slide.

The invention is now described by way of example with reference to the accompanying diagrammatic drawings and a working example.

In the drawings,

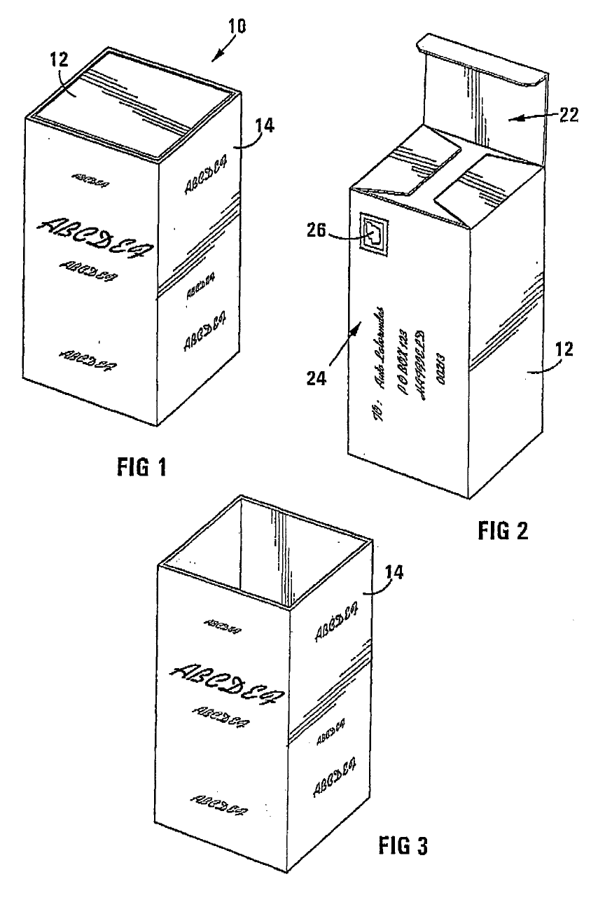

FIG. 1 shows, in three-dimensional view, a do-it-yourself kit in accordance with the invention;

FIG. 2 shows, in three-dimensional view, a holder forming part of the do-it-yourself kit of FIG. 1;

FIG. 3 shows, in three-dimensional view, a sleeve forming part of the do-it-yourself kit of FIG. 1;

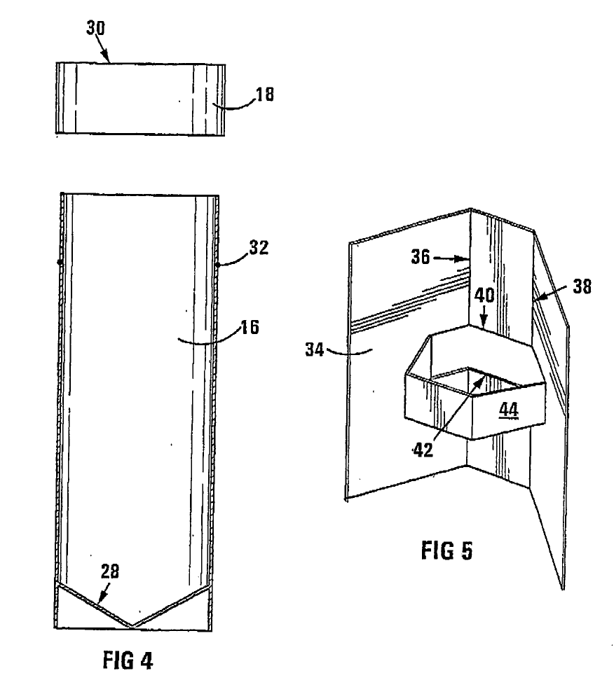

FIG. 4 shows, in axial sectional view, a container forming part of the do-it-yourself kit of FIG. 1; and

FIG. 5 shows, also in three-dimensional view, a securing member also forming part of the do-it-yourself kit of FIG. 1.

Conventionally, women have to undergo a full internal examination by a doctor or a nurse in order to have a pap smear. In accordance with this invention, briefly, it is now possible for women to collect a pap smear sample themselves without having to go through the embarrassment of an internal examination. The procedure relies on the fact that the female body naturally sheds cellular material in the vagina, which material is drawn downward by gravity and which material can thus be absorbed by a tampon.

The woman or client logs onto a website as herein described and receives educational information about the pap smear. She registers her demographic and health information, pays for the smear via electronic payment means, and is able to view a short demonstration video or clip of how to obtain a cytological sample. She can then either order a do-it-yourself kit 10 (FIG. 1) via the website, in which case she pays for it by electronic payment means, or order it directly from a pathological unit or laboratory, or at her own leisure pick up the kit 10 at a wide range of distribution points. The kit 10 may also be dispensed by health care providers. She then collects the pap smear sample using a tampon supplied as part of the kit 10, or her own tampon, in a procedure described in more detail hereinafter and returns the kit 10 either by return mail or to the same point at which she picked it up. Upon receipt of the kit 10, the pathological unit or laboratory analyses the sample collected by the woman (the analysis procedure is described in more detail hereinafter). The result of the analysis will be available online within 4 days of the receipt of the sample. Only if there is an abnormality detected will the woman. then be referred for evaluation by a gynaecologist or other medical practitioner.

The do-it-yourself kit 10, which is made available to the client includes, by way of example, a holder 12 (shown in FIG. 2 and described in more detail hereinafter) contained in a sleeve 14 (shown in FIG. 3 and described in more detail hereinafter), the holder 12 containing:

One package insert describing the procedure in detail, i.e. the insert is in the form of user instructions;

One tampon (mini size) having a retracting cord;

One container in the form of a centrifuge tube 16 sealable by a lid 18 (both shown in FIG. 4 and described in more detail hereinafter) the tube 1 6 containing

25 millilitre preserving liquid consisting of

A water-miscible alcohol such as Methanol/Ethanol (30-50% per volume)

Anti-clamping agent, e.g. Ethylenediaminetetra—acetic acid (EDTA) (2-4% per volume)

Antibiotics:

Vancomycin: 4 micrograms/millilitre

Amikacin: 32 micrograms/millilitre

Amphotericin B: 400 micrograms/millilitre.

Buffer, e.g. Phosphate buffered saline (PBS)

One patient information sheet for receiving information such as demographic, health, and preferred payment option information of the client and information about the client's doctor. It is to be appreciated that if the client ordered the kit 10 via the Internet, the information would already be captured.

The preserving liquid facilitates rapid fixation of live cells, which, in turn, facilitates subsequent analysis of the fixated cells. The liquid also preserves the morphology of cellular matter collected in situ by a tampon in accordance with the invention. The preserving liquid will. typically preserve cellular matter for a period of up to 6 weeks. at temperatures ranging from 4-38° C. The antibiotics will inhibit growth of bacteria or fungus present in the sample, and the buffer will ensure that the pH of the sample remains between 4 and 7.

By way of addition, the kit may further include:

Two glass microscope slides.

A fixative in the form of an atomiser spray for fixating a sample or samples smeared onto the slides.

The user instructions may, for example, be as follows:

To Insert the Tampon:

1. Wash hands thoroughly and remove the wrapper from the tampon;

2. Relax and get into a comfortable position. With the index finger, push the tampon well into the vagina;

the cord will hang outside the vagina ready for removal;

if the tampon can still be felt it needs to be pushed further into the vagina.

3. Wash hands again.

4. Keep the tampon intact for three to eight hours.

To Remove the Tampon (After Three to Eight Hours):

VERY IMPORTANT: Handle the tampon only by the cord.

1. Gently pull out the tampon by only handling the withdrawal cord.

2. Place the tampon into included container, filled with preserving liquid. Close lid tightly and replace container into holder.

3. Wash hands again.

4. Ensure that

the information sheet is completed and replaced into the holder.

5. Return all components.

When the kit includes the glass microscope slides-and the fixative, the user instructions may include also specific instructions on how to prepare a sample using the slides and the fixative.

Upon receipt of the kit by the pathological unit or laboratory, the sample is processed by removing the tampon from the container 16 and concentrating the cellular material. Four different samples are then prepared, a first on which a pap smear or cytological test is to be conducted, a second which is to be screened microbiologically and molecularly, a third which is to be subjected to a Human Papilloma Virus (HPV) test, and a fourth which is to be kept in appropriate storage for future use.

The sample on which the pap smear or cytological test is to be conducted, is prepared on a microscope slide. The slide is stained in a manner similar to that used in conventional pap smear tests, after which a microscopic analysis for cellular abnormalities is conducted.

A further slide is prepared for the microbiological and molecular screening. The sample on the slide is dried whereafter it is Gram stained and only thereafter microbiologically and molecularly screened in order to analyse the vaginal bacteria and through that an analysis of the health of the vagina. This screening allows diagnosis of fungal infections (candida) and of bacterial vaginosis (BV) which, however not life threatening, can cause premature labour during pregnancy.

The HPV test includes an ultrasensitive test, namely the Polymerase Chain Reaction (PCR) test, which can detect the DNA of pathogenic bacteria, viruses and parasites. This test is performed by washing the cellular matter to remove the alcohol of the preservation liquid, whereafter DNA is extracted from the cells. The DNA is then used in a PCR reaction to detect possible presence of HPV, the virus which causes cancer of the cervix, DNA. When HPV DNA is present, a further test (e.g. Digene HPV typing test) is performed to determine the type of HPV present. The PCR test is a more sensitive test than the cytological examination of the cells as it can predict women whose pap smear will become abnormal in future. This is not possible with a conventional pap smear.

Once the results of the various tests and analyses are available, they are logged onto a pathological unit or laboratory management system which is linked to the website. The results are then communicated to the client as prearranged, e.g. via the Internet by use of the client's PIN, by e-mail, telephone, facsimile, in person, or the like.

With reference to FIG. 1 of the drawings, the do-it-yourself kit in accordance with the invention is generally indicated by reference numeral 10. As hereinbefore mentioned, the kit 10 includes a holder 1 2, receivable in a sleeve 14. The holder 12 holds a sealable container 1 6. and a securing member 34. The kit includes also a mini-sized tampon having a retracting cord, user instructions, and a client information sheet (all of which are not shown).

Referring now to FIG. 2 of the drawings, the holder 1 2 is in the form of a parallelepipedal cardboard box having a square cross-section. The holder 12 has a flip-top 22. The holder 1 2 serves as a return envelope in which a sample contained in the container 16 is returned to the pathological unit or laboratory. The address of the pathological unit or laboratory is pre-printed or otherwise marked onto a major face 24 of the holder 12. If the kit is to be returned by mail service, a postage stamp 26 is then fixed to the major face 24 of the holder 12. In a preferred embodiment the dimensions of the holder 12 are 95 mm×46 mm×46 mm.

FIG. 3 shows the sleeve 14, in which the holder 12 is snugly receivable. Upon return of the kit, the sleeve 14 is removed from the holder 12, thereby to expose the return address printed on the major face 24 of the holder 12. Typically, a trade name, or the like, under which the do-it-yourself kit 10 is marketed is printed or otherwise marked onto an outer surface of the sleeve 14.

FIG. 4 shows the sealable container 1 6 with its lid 1 8. The container 1 6 is in the form of a centrifuge tube providing a conical downwardly depending base 28. In use, the sample containing tampon is receivable within the container 1 6. The particular shape of the base 28 facilitates concentration of cellular material from the sample. The lid 1 8 is In the form of a round cylindrical cap having one closed end 30. Close to a top of the container 1 6 it has a circumferentially extending O-ring 32 which, in use, peripherally engages an inner surface of the lid 1 8 thereby to seal the container 16. The container 16 and the lid 18 may be configured to engage each other screw-threadedly and are of a synthetic plastics material, typically polypropylene, which renders it resistant against easy breakage. In use, the preserving liquid (not shown) is contained in the container 1 6, and after the sample has been taken the tampon is inserted into the container 1 6.

The securing member 34, shown in FIG. 5, is in the form of a rectangular cardboard sheet providing two transversely spaced, longitudinally extending fold lines 36, 38. Two longitudinally spaced incisions 40, 42 extend transversely across the securing member 34 and extend outwardly beyond the respective fold lines 36, 38. In use, the securing member 34 is folded along the fold lines 36, 38 to form a shape which, in outline in plan view, is roughly U-shaped. Upon folding of the securing member 34, a section 44 of the securing member 34 defined between the incisions 40, 42 is folded inward into the U-shape, and defines a retaining formation within which the container 1 6 is receivable in use. When folded, the securing member 34 is shaped and dimensioned snugly to be receivable within the holder 1 2. The user instructions and the client information sheet is receivable in the holder 12 inbetween its walls and the retaining formation. In use, the securing member 34 serves also to reinforce the holder 12.

Conventionally, pap smear samples are prepared by scraping cellular material from the cervix of a patient by means of either a wooden Ayres spatula, or a cervical brush or both. The sample is then transferred onto the surface of a slide. The transferral often causes cells to be rolled up, or to lie on top of each other, thus obscuring some of the cells or parts of some of the cells. Also blood and mucus may be transferred to the slide resulting in further obscuring of some of the cells or parts of some of the cells. This invention allows even distribution of cellular material onto a slide with little or no obscuring blood and mucus present. This, in turn, facilitates easier screening or analysis of the sample.

1. A method of providing a pathological service for analysing an exfoliative cytological sample from fluid from reproductive organs of a female (pap smear), including
offering on the Internet by means of a server a pathological service as described;
receiving and processing acceptance of the offer by a client;
making available to the client a do-it-yourself kit for obtaining a cytological sample as described;
receiving the sample;
analysing the sample pathologically; and
communicating the result of the analysis.

2. A method as claimed in claim 1, in which offering of the service on the Internet is via a website.

3. A method as claimed in claim 1, in which receiving and processing acceptance of the offer by the client is by means of an interactive procedure managed by software provided for this purpose in the server.

4. A method as claimed in claim 1, which includes allocating to the client a personal identification number (PIN) to ensure confidentiality.

5. A method as claimed in claim 4, in which communicating the result of the analysis includes making the result available via the server in response to receipt of the PIN.

6. A method as claimed in claim 1, in which receiving acceptance of the offer is personally by telephone, short message service (SMS), facsimile, Internet, email, post, or in person.

7. A method as claimed in claim 1, in which the result of the analysis is personally communicated by telephone, SMS, facsimile, Internet, e-mail, post, or in person.

8. A method as claimed in claim 1, which includes receiving and processing an instruction to provide the result to an interested third party.

9. A method as claimed in claim 1, in which the making available to the client of the do-it-yourself kit is by means of online commerce.

10. A method as claimed in claim 1, in which the making available to the client of the do-it-yourself kit is by means of direct retail or mail order.

11. A method as claimed in claim 1, in which analysing the sample pathologically includes conducting a pap smear test, a Human Papilloma Virus test, and a microbiological and molecular screen of organisms present in the sample.

12. A service facility for providing a pathological service for analysing an exfoliative cytological sample from fluid from reproductive organs of a female (pap smear), the service facility including
a server accessible via the Internet by a client computer, the server having a database incorporating information about the service and having an interactive facility for use by the client computer to obtain a do-it-yourself kit for obtaining a sample as described;
a stock of do-it-yourself kits for obtaining samples as described;
a receiving unit for receiving a sample;
a pathological analysis unit for pathologically analysing the sample; and
a processing and communicating facility of the server for processing a result of the pathological analysis and communicating the result to the client computer via a datalink.

13. A service facility as claimed in claim 12, in which the server makes the database and the interactive facility available via an Internet website.

14. A service facility as claimed in claim 12, in which the interactive facility includes software for receiving particulars of a credit card or like electronic payment means.

15. A service facility as claimed in claim 12, in which the interactive facility has software for allocating a personal identification number (PIN) to a client computer or for receiving a PIN selected by the client and for keeping information about the client confidential against appropriate use of the PIN.

16. A service facility as claimed in claim 15, in which the software is usable to allow verification by means of the PIN for providing a do-it-yourself kit.

17. A service facility as claimed in claim 12, in which the database includes instructions for instructing a client to obtain a sample.

18. A service facility as claimed in claim 17, in which the instructions are in the form of an animation sequence.

19. A do-it-yourself kit for obtaining an exfoliative cytological sample from fluid from reproductive organs of a female (pap smear), the do-it-yourself kit including
a holder;
user instructions in or on the holder for using the kit; and
a sealable container in which a tampon is receivable and which contains a preserving liquid for preserving cellular, bacterial and fungal matter on the tampon after the sample has been obtained, the container being receivable in the holder.

20. A do-it-yourself kit as claimed in claim 19, which includes an information sheet for receiving personal information of a user of the kit.

21. A do-it-yourself kit as claimed in claim 19, in which the preserving liquid includes a pH buffer.

22. A do-it-yourself kit as claimed in claim 19, in which the preserving liquid includes antibiotics.

23. A do-it-yourself kit as claimed in claim 19, in which the preserving liquid includes an anti-clumping agent.

24. A do-it-yourself kit as claimed in claim 19, in which the holder includes securing means for securing the container in position inside the holder.

25. A do-it-yourself kit as claimed in claim 19, which includes a tampon.

26. A do-it-yourself kit as claimed in claim 19, which includes at least one microscope slide and a fixative for fixing a sample smeared onto the slide.

27. A method of providing a pathological service for analysing an exfoliative cytological sample from fluid from reproductive organs of a female (pap smear), including
making available to a client a do-it-yourself kit for obtaining a cytological sample as described;
receiving the sample;
analysing the sample pathologically; and
communicating the result of the analysis.

28. A method as claimed in claim 27, in which the do-it-yourself kit is a kit as claimed in claim 19.

29. A method as claimed in claim 1 or claim 27, substantially as herein described and illustrated.

30. A service facility as claimed in claim 12, substantially as herein described and illustrated.

31. A do-it-yourself kit as claimed in claim 19, substantially as herein described and illustrated.

32. A new method, a new service facility, or a new do-it-yourself kit, substantially as herein described.
